(19) United States
(12) Patent Application Publication
(10) Pub. No.: US 2011/0224558 A1
(43) Pub. Date: Sep. 15, 2011

(54) BLOOD PRESSURE INFORMATION MEASUREMENT DEVICE FOR MEASURING PULSE WAVE PROPAGATION SPEED AS BLOOD PRESSURE INFORMATION

(75) Inventors: Tatsuya Kobayashi, Otsu-shi (JP); Hironori Sato, Moriyama-shi (JP); Toshihiko Ogura, Kyoto-shi (JP); Hideaki Yoshida, Kyoto-shi (JP); Kenji Fujii, Kyoto-shi (JP)

(73) Assignee: OMRON HEALTHCARE Co., Ltd., Kyoto-shi (JP)

(21) Appl. No.: 13/112,398

(22) Filed: May 20, 2011

Related U.S. Application Data
(63) Continuation of application No. PCT/JP2009/069270, filed on Nov. 12, 2009.

(30) Foreign Application Priority Data
Nov. 20, 2008 (JP) 2008-296833

(51) Int. Cl. A61B 5/022

(52) U.S. Cl. 600/490

(57) ABSTRACT

In the measurement device, the blood pressure of the upper arm and the blood pressure of the lower limb are measured using cuffs attached to the upper arm and the lower limb (ankle), respectively. The pulse wave of the upper arm and the pulse wave of the lower limb are measured in synchronization using such cuffs. The upper arm-lower limb pulse wave propagation velocity (baPWV) is calculated based on the appearance time difference of the two pulse waves. The upper arm pulse wave propagation velocity (upper arm PWV) is calculated based on the appearance time difference of the ejection wave and the reflection wave in the upper arm pulse wave. If the values of such propagation velocities are different, a warning is issued.

### TECHNICAL FIELD

The present invention relates to blood pressure information measurement devices, and in particular, to a blood pressure information measurement device for analyzing a pulse wave and calculating an index useful for diagnosis.

### BACKGROUND ART

A device for calculating the velocity of propagation of the pulse wave ejected from the heart (hereinafter referred to as PWV: Pulse Wave Velocity) as an index useful for the diagnosis of the degree of arterial sclerosis has been conventionally proposed. Japanese Unexamined Patent Publication No. 2000-316821 discloses a technique of screening the presence of stenosis of femoral artery and the like by measuring a ratio of the blood pressure of the upper arm and the blood pressure of the ankle (hereinafter referred to as ABI: Ankle-brachial index).

The upper arm and lower limb pulse wave propagation velocity (hereinafter referred to as ba (brachial-ankle) PWV) is calculated by attaching a cuff or the like for measuring the pulse wave on at least two areas of the upper arm, the lower limb and the like and simultaneously measuring the pulse wave to calculate from the appearance time difference of the respective pulse wave and the length of the artery between two points on which the cuff or the like for measuring the pulse wave is attached.
Patent Document 1: Japanese Unexamined Patent Publication No. 2000-316821

### SUMMARY OF INVENTION

If the femoral artery, or the like has stenosis, the blood pressure on the peripheral side lowers, and the baPWV cannot be correctly measured. Thus, it is known that attention needs to be paid to the value of the ABI when measuring the baPWV. If the ABI is smaller than or equal to 0.9, the baPWV cannot be correctly evaluated. Thus, the evaluation of the arterial sclerosis by a different method is necessary in a patient with stenosis, which is a load on both the patient and the measurer.

Therefore, one or more embodiments of the present invention provides a blood pressure information measurement device capable of correctly measuring the pulse wave propagation velocity by separating the ejection wave and the reflection wave from the pulse wave measured from the upper arm and estimating the pulse wave propagation velocity from the appearance time of the reflection wave.

According to one or more embodiments of the present invention, a blood pressure information measurement device includes a first cuff that includes a first air bladder and that is to be attached to an upper arm; a second cuff that includes a second air bladder and that is to be attached to a lower limb; a measurement unit for measuring a change in inner pressure of the first air bladder and change in inner pressure of the second air bladder in synchronization; a detection unit for detecting first blood pressure information from the change in inner pressure of the first air bladder, and detecting second blood pressure information from the change in inner pressure of the second air bladder; and a calculation unit for performing a calculation process based on the first blood pressure information and the second blood pressure information; wherein the calculation unit executes a first calculation process of calculating a first pulse wave propagation velocity from a pulse wave or the first blood pressure information detected from the change in inner pressure of the first air bladder with the first air bladder attached to the upper arm and with blood on a peripheral side of the first cuff stopped and a pulse wave or the second blood pressure information, and a determination process of determining the appropriateness of the first pulse wave propagation velocity calculated by the first calculation process using at least one blood pressure information of the first blood pressure information or the second blood pressure information.

According to one or more embodiments of the present invention, the pulse wave propagation velocity can be measured irrespective of the progress status of the arterial sclerosis.

### BRIEF DESCRIPTION OF THE DRAWINGS

FIG. 1 is a perspective view showing a specific example of an outer appearance of a measurement device according to first to third embodiments.

FIG. 2A is a schematic cross-sectional view showing a measurement position of when measuring blood pressure information using the measurement device shown in FIG. 1.

FIG. 2B is a schematic cross-sectional view showing a measurement position of when measuring blood pressure information using the measurement device shown in FIG. 1.

FIG. 3 is a view showing function blocks of the measurement device according to a first embodiment.

FIG. 4 is a flowchart showing a first specific example of the measurement operation in the measurement device according to the first embodiment.

FIG. 5 is a flowchart showing a second specific example of the measurement operation in the measurement device according to the first embodiment.

FIG. 6 is a flowchart showing a third specific example of the measurement operation in the measurement device according to the first embodiment.

FIG. 7 is a view showing a display example of when the measurement operation according to the third specific example is carried out in the measurement device according to the first embodiment.

FIG. 8 is a view showing function blocks of a measurement device according to a second embodiment.

FIG. 9 is a flowchart showing a specific example of the measurement operation in the measurement device according to the second embodiment.

FIG. 10 is a flowchart showing a specific example of a measurement operation in a measurement device according to a third embodiment.

### DETAILED DESCRIPTION OF INVENTION

Embodiments of the present invention will be described below with reference to the drawings. In the following description, the same reference numerals denote the same components and constituent elements. The names and functions of these components and constituent elements are the same.

The “blood pressure information” refers to the information related to blood pressure that can be obtained by measuring from a living body and specifically includes blood pressure value, pulse wave waveform, heart rate, and the like.

### First Embodiment

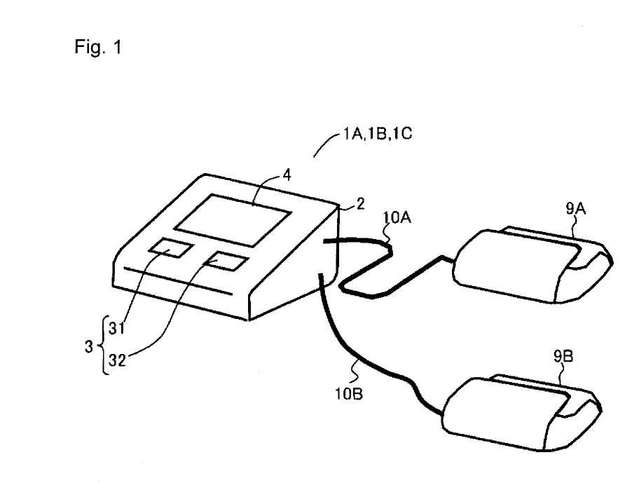

As shown in FIG. 1, a blood pressure information measurement device (hereinafter referred to as measurement device) 1A according to the embodiment includes a base body 2, a cuff 9A to be attached to the upper arm or the measurement site connected to the base body 2 through an air tube 10A, and a cuff 9B to be attached to the lower limb (ankle) or the measurement site connected to the base body 2 through an air tube 10B. A display unit 4 for displaying various information including the measurement result, and an operation unit 3 operated to give various instructions to the measurement device 1A are arranged on a front surface of the base body 2. The operation unit 3 includes a power switch 31 operated to turn ON/OFF the power, and a measurement start switch 32 operated to instruct the start of measurement.

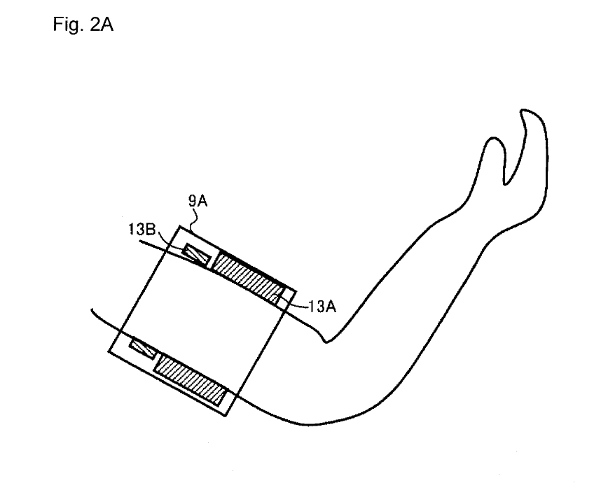

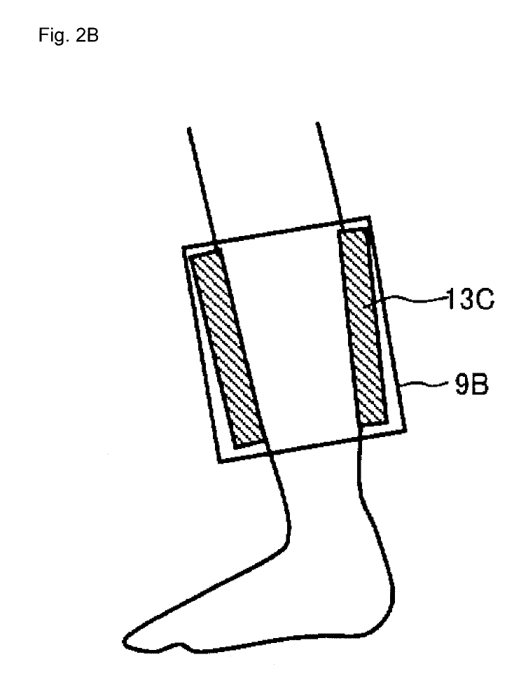

The cuff 9A is wrapped around the upper arm or the measurement site, as shown in FIG. 2A, when measuring the pulse wave using the measurement device 1A. Furthermore, the cuff 9B is wrapped around the lower limb or the measurement site, as shown in FIG. 2B. The blood pressure information is measured when the measurement start switch 32 is pushed in such state.

With reference to FIG. 2A, the cuff 9A includes an air bladder serving as a fluid bladder for compressing the living body. The air bladder includes an air bladder 13A or the fluid bladder used to measure the blood pressure serving as the blood pressure information, and an air bladder 13B or the fluid bladder used to measure the pulse wave serving as the blood pressure information. With reference to FIG. 2B, the cuff 9B includes an air bladder 13C or the fluid bladder used to measure the blood pressure and the pulse wave serving as the blood pressure information.

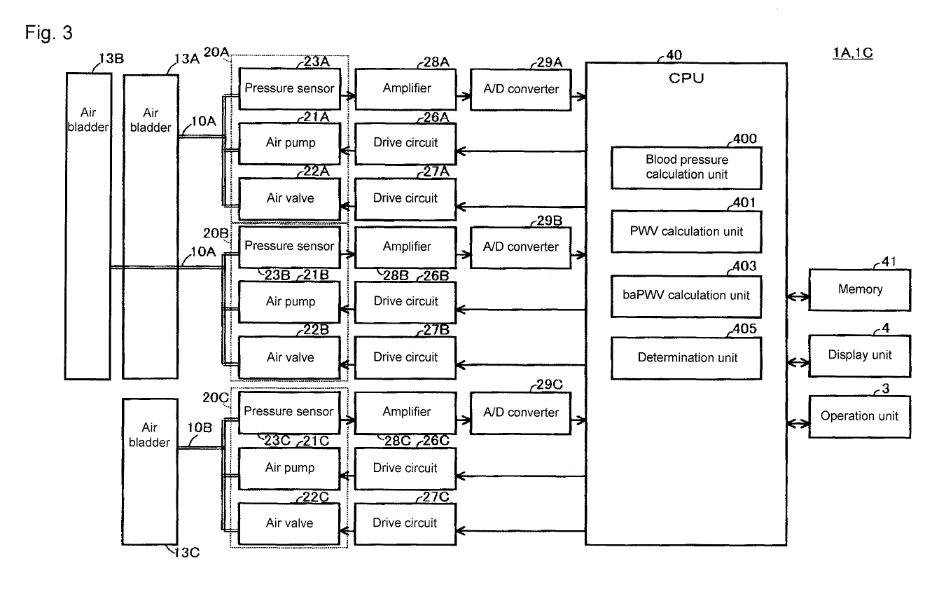

With reference to FIG. 3, the measurement device 1A includes an air system 20A connected to the air bladder 13A through the air tube 10A, an air system 20B connected to the air bladder 13B through the air tube 10A, an air system 20C connected to the air bladder 13C through the air tube 10B, and a CPU (Central Processing Unit) 40.

The air system 20A includes an air pump 21A, an air valve 22A, and a pressure sensor 23A. The air system 20B includes an air pump 21B, an air valve 22B, and a pressure sensor 23B. The air system 20C includes an air pump 21C, an air valve 22C, and a pressure sensor 23C.

The air pump 21A pressurizes the air bladder 13A by being driven by a drive circuit 26A receiving a command from the CPU 40 and sending compressed gas to the air bladder 13A. The air pump 21B pressurizes the air bladder 13B by being driven by a drive circuit 26B receiving a command from the CPU 40 and sending compressed gas to the air bladder 13B. The air pump 21C pressurizes the air bladder 13C by being driven by a drive circuit 26C receiving a command from the CPU 40 and sending compressed gas to the air bladder 13C.

The air valve 22A maintains or depressurizes the pressure of the air bladder 13A by having the open/close state controlled by a drive circuit 27A receiving a command from the CPU 40. The air valve 22B maintains or depressurizes the pressure of the air bladder 13B by having the open/close state controlled by a drive circuit 27B receiving a command from the CPU 40. The air valve 22C maintains or depressurizes the pressure of the air bladder 13C by having the open/close state controlled by a drive circuit 27C receiving a command from the CPU 40. The pressure of the air bladder 13A, 13B, 13C is controlled by controlling the open/close state of the air valves.

The pressure sensor 23A detects the pressure in the air bladder 13A and outputs a signal corresponding to the detected value to an amplifier 28A. The amplifier 28A amplifies the signal output from the pressure sensor 23A and outputs the same to an A/D converter 29A. The A/D converter 29A digitizes the analog signal output from the amplifier 28A and outputs to the CPU 40.

The pressure sensor 23B detects the pressure in the air bladder 13B and outputs a signal corresponding to the detected value to an amplifier 28B. The amplifier 28B amplifies the signal output from the pressure sensor 23B and outputs the same to an A/D converter 29B. The A/D converter 29B digitizes the analog signal output from the amplifier 28B and outputs to the CPU 40.

The pressure sensor 23C detects the pressure in the air bladder 13C and outputs a signal corresponding to the detected value to an amplifier 28C. The amplifier 28C amplifies the signal output from the pressure sensor 23C and outputs the same to an A/D converter 29C. The A/D converter 29C digitizes the analog signal output from the amplifier 28C and outputs to the CPU 40.

The CPU 40 controls the air systems 20A, 20B, 20C based on the command input to the operation unit 3 arranged on the base body 2 of the measurement device. The measurement result is output to the display unit 4 and a memory 41. The memory 41 stores measurement results, and programs to be executed by the CPU 40.

The CPU 40 includes a blood pressure calculation unit 400, a PWV calculation unit 401, a baPWV calculation unit 403, and a determination unit 405. These functions may be functions realized as software when the CPU 40 reads out and executes the program stored in the memory 41, or may be functions realized as hardware when the CPU 40 includes a calculation circuit, and the like.

The blood pressure calculation unit 400 calculates the systolic blood pressure and the diastolic blood pressure at the upper arm and the lower limb from the change in inner pressure of the air bladder 13A and the air bladder 13C.

The PWV calculation unit 401 calculates the appearance time difference of the ejection wave and the reflection wave in the pulse wave from the change in inner pressure of the air bladder 13B in a state the blood is stopped at the peripheral side by the air bladder 13A. The PWV calculation unit 401 calculates the propagation velocity of the pulse wave at the upper arm (upper arm PWV) by dividing the length of the artery from the heart to the upper arm stored in advance by the time difference.

The baPWV calculation unit 403 calculates the appearance time difference of the pulse wave at the upper arm and the lower limb from the change in inner pressure of the air bladder 13B and the air bladder 13C. The baPWV calculation unit 403 calculates the propagation velocity of the pulse wave at the upper arm and the lower limb (baPWV) by dividing the difference in the length of the artery from the heart to the ankle and the length of the artery from the heart to the upper arm stored in advance by the time difference.

The length of the artery from the heart to the upper arm and the length of the artery from the heart to the ankle used in the PWV calculation unit 401 and the baPWV calculation unit 403 may be stored in the PWV calculation unit 401 and the baPWV calculation unit 403 in advance, or may be calculated from the input height of the person being measured by the PWV calculation unit 401 and the baPWV calculation unit 403 using a predetermined conversion equation.

The determination unit 405 compares the PWV calculated in the PWV calculation unit 401 and the baPWV calculated in the baPWV calculation unit 403 to determine success or fail of the measurement of the baPWV. The CPU 40 executes a process for causing the display unit 4 to display the measurement result or a process for causing the display unit 4 to display a display warning that the measurement failed depending on the determination result of the determination unit 405.

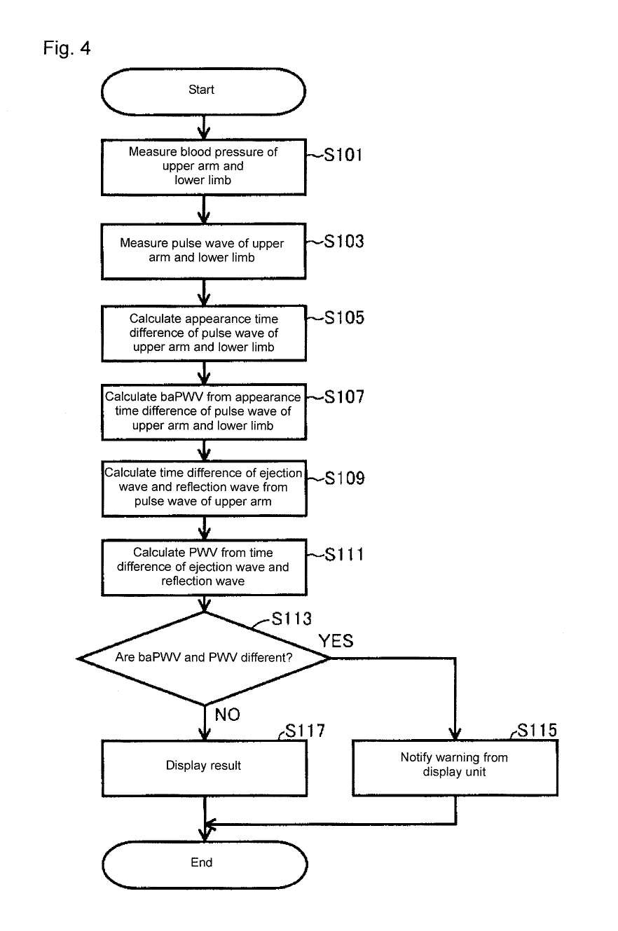

A first specific example of the measurement operation in the measurement device 1A of FIG. 4 shows the measurement operation of when the calculation with the first calculation algorithm is carried out. The operation shown in FIG. 4 is started when the measurer pushes the measurement button arranged on the operation unit 3 of the base body 2, and is realized when the CPU 40 reads out the program stored in the memory 41 and controls each unit shown in FIG. 3.

With reference to FIG. 4, the CPU 40 outputs a control signal for carrying out the blood pressure measurement to each air system 20A, 20B, and 20C in step S101. The blood pressure calculation unit 400 calculates the blood pressure value at the upper arm and the blood pressure value at the lower limb based on the change in inner pressure obtained from the air bladder 13A and the air bladder 13C. The blood pressure measurement operation here is similar to the measurement operation in the normal blood pressure measurement device.

In step S103, the CPU 40 outputs a control signal for carrying out the pulse wave measurement to each air system 20A, 20B, 20C, and measures the pulse wave at the upper arm and the pulse wave at the lower limb in synchronization. In step S103, the CPU 40 causes the drive circuits 26A, 27A to supply air with the air pump 21A to maintain the inner pressure of the air bladder 13A at a pressure higher than the systolic blood pressure value, and outputs a control signal for closing the air valve 22A so that the blood is stopped at the peripheral side of the upper arm. The control signal for causing the drive circuits 26B, 26C to supply air so that the inner pressures of the air bladder 13B and the air bladder 13C are the pressures of an extent the pulse beat can be felt defined in advance is then output in this state, and the pulse wave of the upper arm and the pulse wave of the lower limb are obtained in synchronization based on the pressure signals obtained from the pressure sensors 23B, 23C. The way of synchronizing is not limited to a specific method.

In step S105, the baPWV calculation unit 403 analyzes the pulse wave of the upper arm and the pulse wave of the lower limb obtained in step S103, and calculates the appearance time difference from the difference in the rising point of the pulse waves. In step S107, the baPWV calculation unit 403 calculates the propagation velocity of the pulse wave (baPWV) by dividing the difference in the length of the artery from the heart to the ankle and the length of the artery from the heart to the upper arm stored in advance by the time difference calculated in step S105.

In step S109, the PWV calculation unit 401 analyzes the pulse wave of the upper arm obtained in step S103, and calculates the appearance time difference of the difference in the appearance time of the ejection wave and the appearance time of the reflection wave in the relevant pulse wave. In step S111, the PWV calculation unit 401 calculates the propagation velocity of the pulse wave at the upper arm (upper arm PWV) by dividing the length of the artery from the heart to the upper arm stored in advance by the time difference calculated in step S109.

In step S113, the determination unit 405 compares the baPWV calculated in step S107 and the upper arm PWV calculated in step S111, and determines whether they are the same or different. When referring to “same”, this is not limited to being completely the same and also includes a case of being within a range of a certain extent. The “range of a certain extent” may be about 200 cm/s. If it is determined in step S113 that the baPWV and the upper arm PWV are different (or have a difference of greater than or equal to a range of a certain extent) (YES in step S113), the CPU 40 executes the process of causing the display unit 4 to display a warning notifying that the measurement of the baPWV failed in step S115. If it is determined in step S113 that the baPWV and the upper arm PWV are the same (or within the range of a certain extent) (NO in step S113), the CPU 40 causes the display unit 4 to display the blood pressure value calculated in step S101 and the baPWV calculated in step S107 as measurement results in step S117 assuming the measurement of the baPWV was successful.

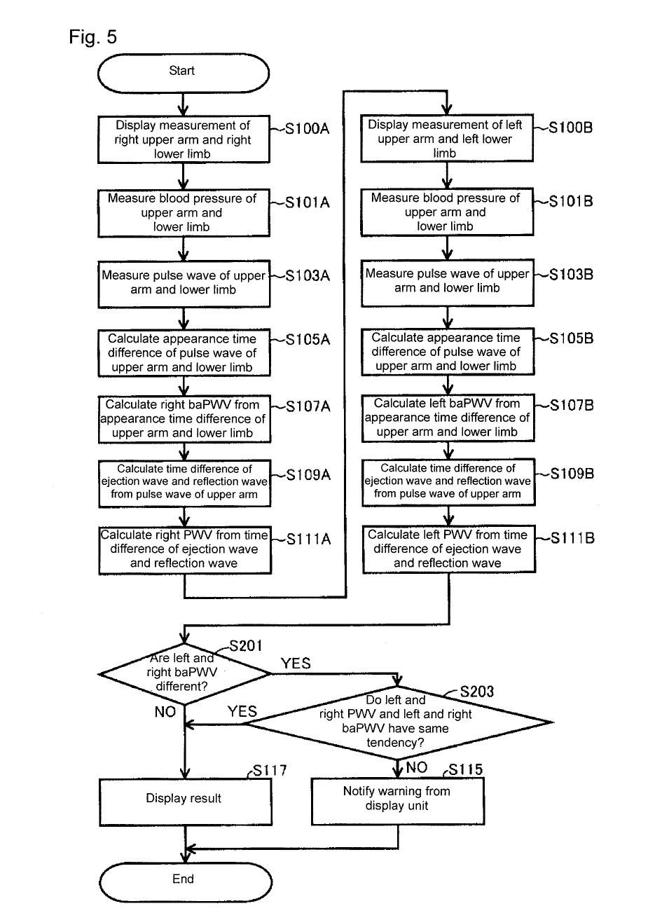

A second specific example of the measurement operation in the measurement device 1A of FIG. 5 shows the measurement operation of when the calculation with the second calculation algorithm is carried out. The operation shown in FIG. 5 is also started when the measurer pushes the measurement button arranged on the operation unit 3 of the base body 2, and is realized when the CPU 40 reads out the program stored in the memory 41 and controls each unit shown in FIG. 3.

With reference to FIG. 5, the measurement shown in steps S101 to S111 and the calculation operation of the baPWV and the upper arm PWV are carried out for the left and the right in the measurement operation according to the second specific example. In other words, the operations similar to steps S101 to S111 of the operation according to the first specific example are carried out with the cuffs 9A, 9B attached to the right upper arm and the right lower limb (right ankle) in steps S101A to S111A. Thereafter, the operations similar to steps S101 to S111 of the operation according to the first specific example are carried out with the cuffs 9A, 9B attached to the left upper arm and the left lower limb (left ankle) in steps S101B to S111B. Thus, according to one or more embodiments of the present invention, the CPU 40 causes the display unit 4 to display a notification of attaching the cuffs 9A, 9B respectively to the right upper arm and the right lower limb (right ankle) or the left upper arm and the left lower limb (left ankle) and measuring the blood pressure and the pulse wave thereof to notify to the measurer in steps S100A and S100B prior to each process.

The CPU 40 temporarily stores the baPWV (right baPWV) by the right side measurement result calculated in step S107A, the right upper arm PWV calculated in step S111A, the baPWV (left baPWV) by the left side measurement result calculated in step S107B, and the left upper arm PWV calculated in step S111B separately for the left and for the right. In step S201, the determination unit 405 compares the right baPWV calculated in step S107A and the left baPWV calculated in step S107B, and determines whether they are the same or different. When referring to “same”, this is not limited to being completely the same and also includes a case of being within a range of a certain extent. If it is determined that the right baPWV and the left baPWV are different in step S113 (YES in step S201), the determination unit 405 compares the tendency of the right baPWV and the left baPWV, and the tendency of the right upper arm PWV and the left upper arm PWV in step S203. The tendency referred to here may be the magnitude relationship and difference, or may be the proportion of one with respect to the other. In other words, the tendency refers to the degree of change between the two values, where if there is a correlation between the degree of change from the right baPWV to the left baPWV and the degree of change from the right upper arm PWV to the left upper arm PWV (left and right may be reversed) such as if the degree of change is within a predetermined range, the determination unit 405 determines that the tendency is the same. If the tendencies are different in step S203 (NO in step S203), the CPU 40 executes the process of causing the display unit 4 to display a warning notifying that the measurement of the baPWV failed in step S115. If it is determined that the right baPWV and the left baPWV are the same in step S201 (or within the range of a certain extent) (NO in step S201), the CPU 40 causes the display unit 4 to display the blood pressure value calculated in steps S101A, S101B and the baPWVs calculated in steps S107A, S107B as measurement results in step S117 assuming the measurement of the baPWV was successful. If the tendency of the right baPWV and the left baPWV, and the tendency of the right upper arm PWV and the left upper arm PWV are the same (YES in step S203), the CPU 40 causes the display unit 4 to display the blood pressure value calculated in steps S101A, S101B and the baPWVs calculated in steps S107A, S107B as measurement results in step S117 even if the right baPWV and the left baPWV are different in step S201 assuming it is within the range of the left and right difference unique to the person being measured. The measurement result of either the left or the right may be displayed or of both may be displayed, or an average value thereof may be displayed.

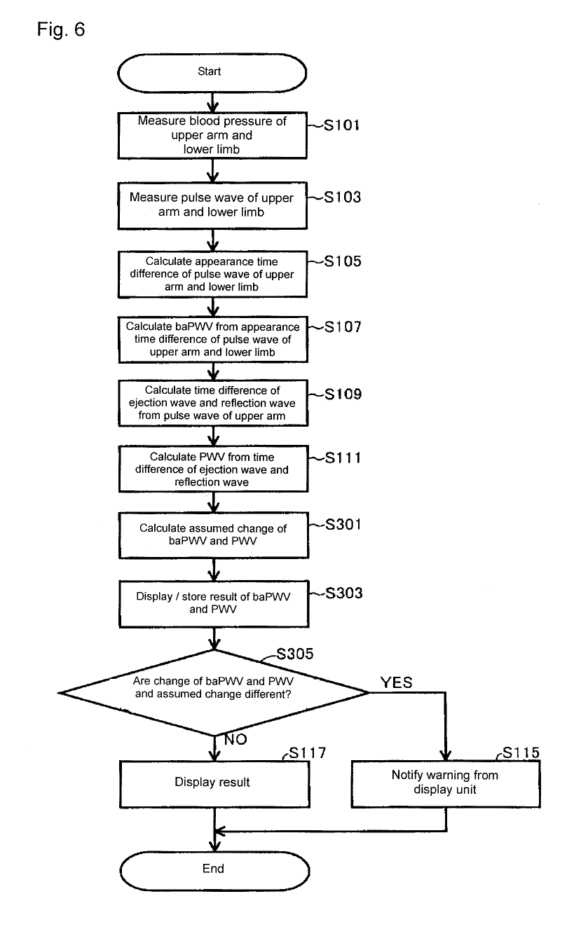

A third specific example of the measurement operation in the measurement device 1A of FIG. 6 shows the measurement operation of when the calculation with the third calculation algorithm is carried out. The operation shown in FIG. 6 is also started when the measurer pushes the measurement button arranged on the operation unit 3 of the base body 2, and is realized when the CPU 40 reads out the program stored in the memory 41 and controls each unit shown in FIG. 3.

With reference to FIG. 6, the same operation as in steps S101 to S111 is carried out, where the pulse wave is measured and the baPWV and the upper arm PWV are calculated in the measurement operation according to the third specific example. In the measurement operation according to the third specific example, the calculated baPWV and the upper arm PWV are stored in a predetermined region of the memory 41 in correspondence with the information such as the measurement date and time, the number of measurement counts, and the like with which at least the anteroposterior relationship of the measurement of other information result and the measurement of this time can be known. After the operation up to step S111 is completed and the baPWV and the upper arm PWV are calculated, the determination unit 405 reads out the baPWV and the upper arm PWV obtained in at least the measurement of the previous time and calculates the baPWV and the upper arm PWV of this time assuming the change of the baPWV and the upper arm PWV obtained in the measurement of the previous time in step S301. The assuming method is not specifically limited, but the change corresponding to a predetermined condition such as performing a certain treatment or administering medicine may be stored in advance in the memory 41, and the determination unit 405 may read out the change corresponding to the input condition from the memory 41 and apply the change with respect to the read baPWV and the upper arm PWV of the previous time to predict the baPWV and the upper arm PWV of this time. Alternatively, a plurality of calculation results of the baPWV and upper arm PWV may be stored in the memory 41, so that the determination unit 405 can calculate the tendencies thereof to predict the baPWV and the upper arm PWV of this time.

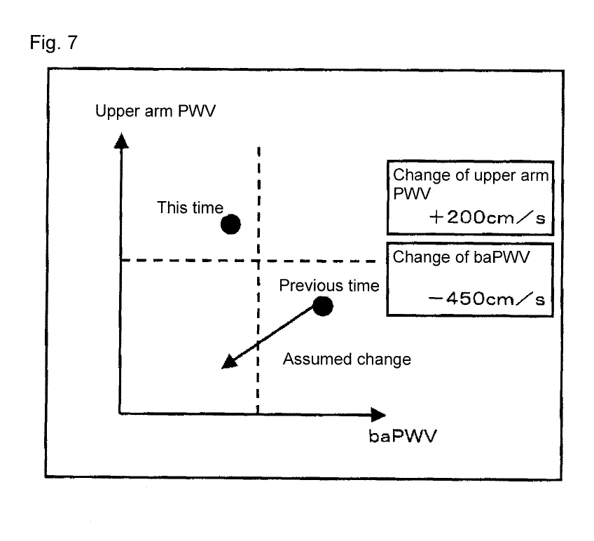

In step S303, the CPU 40 performs the process of causing the display unit 4 to display the baPWV and the upper arm PWV calculated in the operation up to step S111, and stores the same in correspondence with the information with which the anteroposterior relationship at the time of measurement can be recognized such as the measurement date and time for this time in a predetermined region of the memory 41. In this case, the predicted value calculated in step S301 is also displayed on the display unit 4 along with the baPWV and the upper arm PWV obtained in the measurement for this time, as shown in FIG. 7. Thus, whether the baPWV and the upper arm PWV obtained in the measurement for this time are greatly diverged from the change assumed from the measurement of the previous time or are within the assumed range can be easily visually recognized.

In step S305, the determination unit 405 compares the change from the measurement result of the previous time of the baPWV and the upper arm PWV calculated in the operation up to step S111, and the change assumed from the measurement of the previous time calculated in step S301, and determines whether they are the same or different. When referring to “same”, this is not limited to being completely the same and also includes a case of being within a range of a certain extent. If it is determined in step S305 that the change from the measurement result of the previous time of the baPWV and the upper arm PWV calculated in the operation up to step S111 is different from the change assumed from the measurement of the previous time calculated in step S301 (or have a difference of greater than or equal to a range of a certain extent) (YES in step S305), the CPU 40 executes the process of causing the display unit 4 to display a warning notifying that the measurement of the baPWV failed in step S115. If it is determined in step S305 that the changes are the same (or within the range of a certain extent) (NO in step S305), the CPU 40 causes the display unit 4 to display the blood pressure value calculated in step S101 and the baPWV calculated in step S107 as measurement results in step S117 assuming the measurement of the baPWV was successful.

If the baPWV and the upper arm PWV obtained from the simultaneously measured pulse wave are different by greater than or equal to a predetermined value, it may be because the measurement error is included or the baPWV is underestimated due to stenosis of femoral artery, and the like. The measurer can recognize the possibility that the measurement error is included or the possibility that the baPWV is underestimated by the stenosis of femoral artery by executing the measurement operation shown as the first specific example in the measurement device 1A.

If the baPWV measured at the right upper arm and the right lower limb and the baPWV measured at the left upper arm and the left lower limb do not have the same tendency, or if the change from the measurement result of the previous time of the baPWV and the upper arm PWV and the change assumed from the measurement of the previous time are different by greater than or equal to a predetermined value, the reason therefor may be that the measurement error is included or the baPWV is underestimated due to stenosis of femoral artery, and the like. The measurer can recognize the possibility that the measurement error is included or the possibility that the baPWV is underestimated by the stenosis of femoral artery by executing the measurement operation shown as the second specific example or the measurement operation shown as the third specific example in the measurement device 1A. Furthermore, even if the baPWV measured at the right upper arm and the right lower limb and the baPWV measured at the left upper arm and the left lower limb are different, if the tendencies thereof are the same as the tendency of the right upper arm PWV and the left upper arm PWV measured at the same time, determination is made that the baPWV is appropriately measured within the range of the left and right difference unique to the person being measured and the measurement result is displayed.

Here, re-measurement is urged by the warning to obtain a more accurate baPWV.

### Second Embodiment

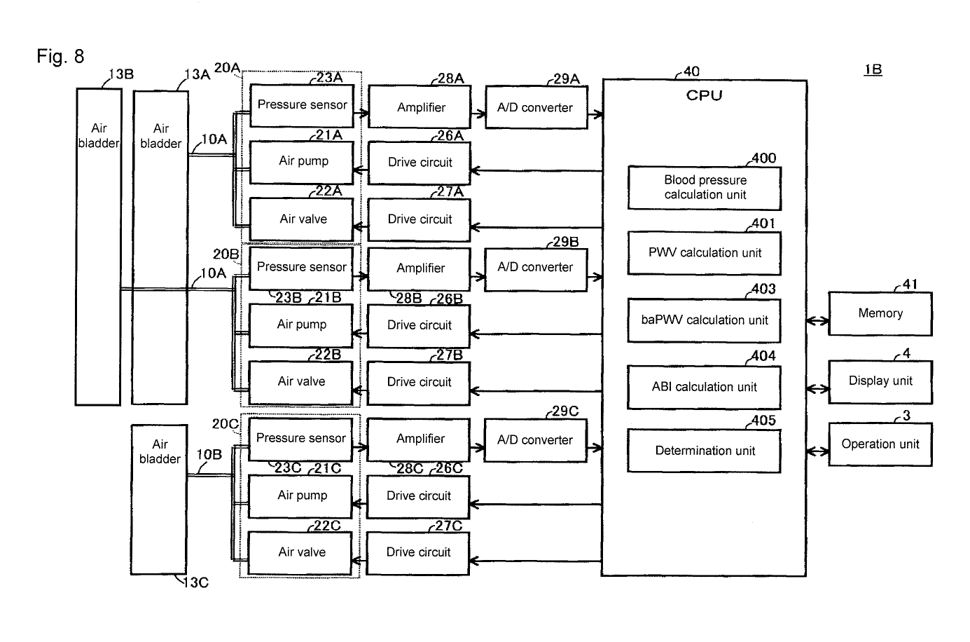

The outer appearance of a measurement device 1B according to a second embodiment is similar to the measurement device 1A shown in FIG. 1. With reference to FIG. 8, the function configuration of the measurement device 1B differs from the function configuration of the measurement device 1A shown in FIG. 3 in that the CPU 40 of the measurement device 1B further includes an ABI calculation unit 404. Such function may also be a function realized as software when the CPU 40 reads out and executes the program stored in the memory 41, or may be functions realized as hardware when the CPU 40 includes a calculation circuit, and the like.

The ABI calculation unit 404 calculates the ABI or the ratio of the blood pressure of the lower limb with respect to the blood pressure value of the upper arm from the blood pressure obtained from the change in inner pressure of the air bladder 13A and the air bladder 13C input in synchronization to the blood pressure calculation unit 400, that is, the blood pressure of the upper arm and the blood pressure of the lower limb calculated at the same time. The determination unit 405 stores the reference value of the ABI in advance, and determines whether to calculate the PWV in the PWV calculation unit 401 or to calculate the baPWV in the baPWV calculation unit 403 by comparing the ABI calculated in the ABI calculation unit 404 and the reference value. The determination unit 405 outputs the control signal for executing the calculation process to the PWV calculation unit 401 or the baPWV calculation unit 403 according to the determination result. The PWV calculation unit 401 executes the calculation of the PWV according to the control signal, and the baPWV calculation unit 403 executes the calculation of the baPWV according to the control signal.

At the normal time, the ABI is within the range of about 0.9 to 1.3, but the ABI becomes lower than such range if the person being measured has symptoms in which the blood pressure on the lower limb side lowers such as the arteriosclerosis obliterans (ASO) in which the artery of the peripheral side (mainly lower limb) chronically closes. In this case, the correct baPWV cannot be obtained, as described earlier. Therefore, according to one or more embodiments of the present invention, the upper arm PWV is calculated in this case as an index that does not use the blood pressure of the lower limb.

The determination unit 405 stores the lower limit value of the range of the ABI, for example, the value of about 0.9 which is the normal range as the reference value, and compares the ABI calculated in the ABI calculation unit 404 with the reference value to determine whether or not the calculated ABI is lower than such range. If not lower, that is, if determined to be within the range and as being normal, the control signal for calculating the baPWV is output to the baPWV calculation unit 403, whereas if lower, the control signal for calculating the PWV is output to the PWV calculation unit 401 without performing the calculation of the baPWV assuming there is a possibility of ASO.

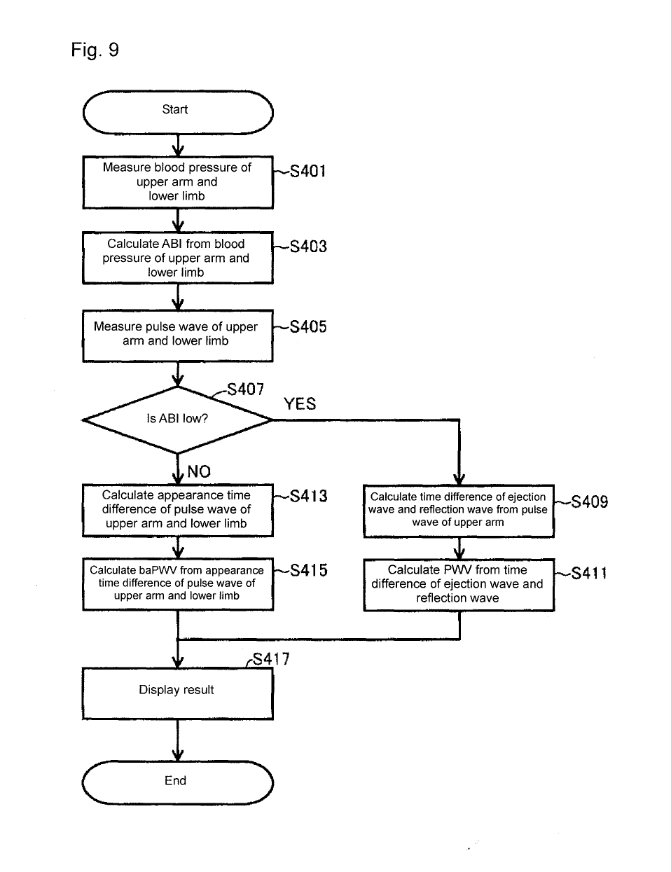

The operation in the measurement device 1B shown in FIG. 9 is also started when the measurer pushes the measurement button arranged on the operation unit 3 of the base body 2, and is realized when the CPU 40 reads out the program stored in the memory 41 and controls each unit shown in FIG. 8.

With reference to FIG. 9, the blood pressure calculation unit 400 calculates the blood pressure value at the upper arm and the blood pressure value at the lower limb in step S401. The operation of measuring the blood pressure at each measurement site is the same as the operation of step S101, but the blood pressure at a plurality of areas is measured in synchronization, that is, at the same timing in step S401. The way of synchronizing is not limited to a specific method. In step S403, the ABI calculation unit 404 calculates the ABI by dividing the blood pressure value of the lower limb measured in step S401 with the blood pressure value of the upper arm. In step S405, the CPU 40 obtains the pulse wave of the upper arm and the pulse wave of the lower limb in synchronization based on the pressure signal obtained from the pressure sensors 23B, 23C. The operation of step S405 is the same as the operation of step S103.

In step S407, the determination unit 405 compares the ABI calculated in step S403 with the stored reference value to determine whether or not the calculated ABI is lower than the reference value. The reference value is a lower limit value of the range of the ABI assumed as the normal range, and is 0.9 in the example. If it is determined that the ABI is lower than the reference (YES in step S407), the determination unit 405 outputs the control signal for executing the calculation process to the PWV calculation unit 401. In step S409, the calculation unit 401 analyzes the pulse wave of the upper arm obtained in step S405 and calculates the appearance time difference or the difference in the appearance time of the ejection wave and the appearance time of the reflection wave in the pulse wave according to the control signal, and calculates the upper arm PWV in step S411. The operation here is the same as steps S109, S111.

If it is determined that the ABI is not lower than the reference, that is, greater than the lower limit value of the range assumed as normal (NO in step S407), the determination unit 405 outputs the control signal for executing the calculation process to the baPWV calculation unit 403. In step S413, the baPWV calculation unit 403 analyzes the pulse wave of the upper arm and the pulse wave of the lower limb obtained in step S405 and calculates the appearance time difference from the difference in the rising points of the pulse waves according to the control signal, and calculates the baPWV in step S415. The operation here is the same as steps S105, S107.

In step S417, the CPU 40 causes the display unit 4 to display the upper arm PWV calculated in step S411 or the baPWV calculated in step S415 as measurement results. In this case, the ABI calculated in step S403 may also be displayed. The relationship between the calculated ABI and the range assumed as normal or the reference value or the lower limit value thereof may also be displayed.

When the measurement operation described above is executed in the measurement device 1B, the upper arm PWV is calculated automatically instead of the baPWV and then displayed if the person being measured has symptoms in which the blood pressure on the lower limb side lowers such as the arteriosclerosis obliterans. The baPWV thus can be prevented from being underestimated when the person has such symptoms.

### Third Embodiment

The outer appearance of a measurement device 1C according to a third embodiment is similar to the measurement device 1A shown in FIG. 1. The function configuration of the measurement device 1C is the same as the function configuration of the measurement device 1A shown in FIG. 3.

The blood pressure calculation unit 400 of the CPU 40 of the measurement device 1C calculates the blood pressure value of the upper arm and the blood pressure value of the lower limb according to the measurement operation, and inputs the same to the determination unit 405. The determination unit 405 determines whether or not the blood pressure of the lower limb is measured by determining whether or not the input value is a value indicating that the blood pressure value is calculated based on the input value from the blood pressure calculation unit 400. If the input value from the blood pressure calculation unit 400 is not a value indicating that the blood pressure value is calculated, that is, the input value determining that the blood pressure of the lower limb is not measured includes a signal value (e.g., 0) indicating that it is not measured, it is not an appropriate value for the blood pressure value of the lower limb (e.g., value not within the range compared to the range of the normal blood pressure value of a human stored in advance), and the like. The determination unit 405 also determines that the blood pressure of the lower limb is not measured if the input of a value indicating the blood pressure value of the lower limb is not made within a predetermined time from the blood pressure calculation unit 400. The factors the blood pressure of the lower limb is not measured may be that the cuff 9B is not appropriately attached to the lower limb (ankle). The CPU 40 outputs the control signal for performing the pulse wave measurement to each air system 20A, 20B, 20C when it is determined that the blood pressure is measured at the lower limb in the determination unit 405.

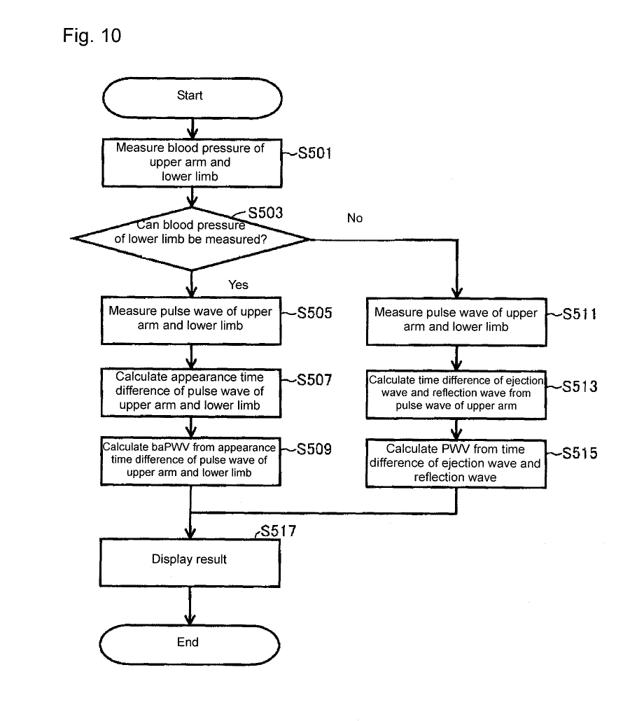

The operation in the measurement device 1B shown in FIG. 10 is also started when the measurer pushes the measurement button arranged on the operation unit 3 of the base body 2, and is realized when the CPU 40 reads out the program stored in the memory 41 and controls each unit shown in FIG. 3.

With reference to FIG. 10, the CPU 40 outputs a control signal for carrying out the blood pressure measurement to each air system 20A, 20B, and 20C in step S501. The blood pressure calculation unit 400 calculates the blood pressure value at the upper arm and the blood pressure value at the lower limb. This blood pressure measurement operation is similar to step S101. In step S503, the determination unit 405 determines whether or not the blood pressure of the lower limb is measured in step S501 based on the input value from the blood pressure calculation unit 400. As described above, if the cuff 9B is not appropriately attached to the lower limb, the blood pressure value of the lower limb cannot be calculated in the blood pressure calculation unit 400, and the value is not input (or value such as 0 indicating that measurement is not appropriately made is input). The determination unit 405 determines that the blood pressure of the lower limb is not measured in step S501 as in such case with reference to the blood pressure value of the lower limb.

If the blood pressure value of the lower limb is calculated in step S501 (YES in step S503), the pulse wave of the upper arm and the pulse wave of the lower limb are measured in steps S505 to S509, similar to steps S103 to S107 or steps S405, S411, S413, and baPWV is calculated by the baPWV calculation unit 403.

If the blood pressure value of the lower limb is not calculated in step S501 (NO in step S503), the CPU 40 causes the drive circuits 26A, 27A to supply air with the air pump 21A and outputs the control signal for closing the air valve 22A to realize a state in which the blood is stopped at the peripheral side of the upper arm to maintain the inner pressure of the air bladder 13A at a pressure higher than the systolic blood pressure in step S511. The control signal for causing the drive circuit 26B to supply air so that the inner pressures of the air bladder 13B becomes a pressure of an extent the pulse beat can be felt defined in advance is then output in this state, and the pulse wave of the upper arm is obtained based on the pressure signal obtained from the pressure sensor 23B. The operation of step S511 is the same as the operation of not performing the operation of measuring the pulse wave of the lower limb and measuring only the pulse wave of the upper arm of the pulse wave measurement operation of step S103 and step S405.

In steps S513, S515, the operations similar to steps S109, S111 or steps S409, S411 are performed, and the upper arm PWV is calculated in the PWV calculation unit 401.

If the blood pressure of the lower limb is not measured when the measurement operation described above is executed in the measurement device 1C, assumption is made that the cuff 9B is not appropriately attached to the lower limb, whereby only the pulse wave of the upper arm is automatically measured and the upper arm PWV is calculated and displayed instead of the baPWV. That is, if there is a possibility the pulse wave of the lower limb is not appropriately measured, only the pulse wave of the upper arm is automatically measured and the upper arm PWV is calculated. Therefore, the calculation of the wrong baPWV can be prevented when the pulse wave of the lower limb is not appropriately measured. Furthermore, the load of the person being measured can be suppressed because the measurement of the pulse wave of the lower limb is not performed at the time point determination is made that the blood pressure of the lower limb is not measured in the blood pressure measurement. The load of the person to be measured can be suppressed because the pulse wave propagation velocity is calculated on the upper arm side without redoing the measurement itself to obtain the pulse wave propagation velocity as an index.

The idea of the measurement device 1C may be applied to the measurement device 1B. In other words, in the measurement device 1B, determination may be made on whether or not the ABI is lower than the standard in the determination unit 405 at the time point the ABI is calculated in step S403, and only the measurement of the pulse wave of the upper arm may be carried out when determined as low. The load of the person to be measured can be suppressed in such manner.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

### DESCRIPTION OF REFERENCE NUMERALS

1A, 1B, 1C measurement device
2 base body
3 operation unit
4 display unit
9A, 9B cuff
10A, 10B air tube
13A, 13B, 13C air bladder
20A, 20B, 20C air system
21A, 21B, 21C air pump
22A, 22B, 22C air valve
23A, 23B, 23C pressure sensor
26A. 26B, 26C, 27A, 27B, 27C drive circuit
28A, 28B, 28C amplifier
29A, 29B, 29C A/D converter
31, 32 switch
40 CPU
41 memory
400 blood pressure calculation unit
401 PWV calculation unit
403 baPWV calculation unit
404 ABI calculation unit
405 determination unit

1. A blood pressure information measurement device comprising:
a first cuff comprising a first air bladder that attaches to an upper arm;
a second cuff comprising a second air bladder that attaches to a lower limb;
a measurement unit that measures a change in inner pressure of the first air bladder and a change in inner pressure of the second air bladder in synchronization;
a detection unit that detects first blood pressure information from the change in inner pressure of the first air bladder, and detects second blood pressure information from the change in inner pressure of the second air bladder; and
a calculation unit that performs a calculation process based on the first blood pressure information and the second blood pressure information,
wherein the calculation unit executes a first calculation process of calculating a first pulse wave propagation velocity from a pulse wave or the first blood pressure information detected from the change in inner pressure of the first air bladder with the first air bladder attached to the upper arm and with blood on a peripheral side of the first cuff stopped and a pulse wave or the second blood pressure information, and
wherein the calculation unit executes a determination process of determining an appropriateness of the first pulse wave propagation velocity calculated by the first calculation process using at least one blood pressure information of the first blood pressure information or the second blood pressure information.

2. The blood pressure information measurement device according to claim 1,
wherein the calculation unit further executes a second calculation process of calculating a second pulse wave propagation velocity from the pulse wave or the first blood pressure information,
wherein the calculation unit calculates an appearance time difference of the pulse wave or the first blood pressure information and the pulse wave or the second blood pressure information to calculate the first pulse wave propagation velocity in the first calculation process, and
wherein the calculation unit calculates an appearance time difference of an ejection wave and a reflection wave in the pulse wave or the first blood pressure information to calculate the second pulse wave propagation velocity in the second calculation process.

3. The blood pressure information measurement device according to claim 2,
wherein the calculation unit compares the first pulse wave propagation velocity calculated in the first calculation process and the second pulse wave propagation velocity calculated in the second calculation process, and determines whether a difference is greater than a predetermined range for the determination of the appropriateness of the first pulse wave propagation velocity calculated in the first calculation process in the determination process, and
wherein the calculation unit performs a first display control for causing a display device to display a warning when the difference is greater than the predetermined range.

4. The blood pressure information measurement device according to claim 2,
wherein the calculation unit further executes a third calculation process of calculating a blood pressure ratio of the blood pressure value or the second blood pressure information with respect to the blood pressure value or the first blood pressure information,
wherein the calculation unit compares the blood pressure ratio with a threshold value stored in advance to determine whether or not the blood pressure ratio is lower than the threshold value for the determination of the appropriateness of the first pulse wave propagation velocity calculated in the first calculation process in the determination process, and
wherein the calculation unit performs a second display control for causing a display device to display the second pulse wave propagation velocity calculated in the second calculation process as a measurement result when the blood pressure ratio is lower than the threshold value.

5. The blood pressure information measurement device according to claim 4,
wherein the calculation unit executes the second calculation process that calculates the second pulse wave propagation velocity when the blood pressure ratio is lower than the threshold value, and executes the first calculation process that calculates the first pulse wave propagation velocity when the blood pressure value is not lower than the threshold value.

6. The blood pressure information measurement device according to claim 2,
wherein the calculation unit determines whether or not the blood pressure value serving as the second blood pressure information is obtained for the determination of the appropriateness of the first pulse wave propagation velocity calculated in the first calculation process in the determination process, and
wherein the calculation unit performs a third display control for causing a display device to display the second pulse wave propagation velocity calculated in the second calculation process as a measurement result when the second blood pressure value is not obtained.

7. The blood pressure information measurement device according to claim 6, further comprising:
a control unit that causes the measurement unit to measure the change in inner pressure of the first air bladder and the calculation unit to perform the second calculation process and to perform the calculation of the second pulse wave propagation velocity from the pulse wave or the first blood pressure information obtained in the measurement if the second blood pressure value is not obtained, and that causes the measurement unit to measure the change in inner pressure of the first air bladder and the change in inner pressure of the second air bladder in synchronization and the calculation unit to perform the first calculation process and perform calculation of the first pulse wave propagation velocity from the pulse wave or the first blood pressure information and the pulse wave or the second blood pressure information obtained in the measurement if the second blood pressure value is obtained.
